(19) United States
(12) Patent Application Publication
(10) Pub. No.: US 2010/0099177 A1
(43) Pub. Date: Apr. 22, 2010

(54) In-situ optical monitoring subsystem compatible with cell incubators

(76) Inventors: Lung-Jieh Yang, Tamsui (TW); Yu-Cheng Ou, Tzu-Kuan (TW)

Correspondence Address: Lung-Jieh Yang, P. O. Box 55-846, Taipei, 104, TW

(21) Appl. No.: 12/319,052

(22) Filed: Jan. 2, 2009

(30) Foreign Application Priority Data
Oct. 20, 2008 (TW) 97140153

(51) Int. Cl. C12M 1/34

(52) U.S. Cl. 435/288.7

(57) ABSTRACT

An optical monitoring subsystem includes: a differential interference contrast (DIC) optical-path apparatus having a CCD camera and enclosed in a gas-tight housing stored in an incubator and a three-dimension electric driving device operatively moving along three axes of three-dimension coordinates for conveniently observing, monitoring or photographing cells in a cell specimen placed in a concave platform as recessed in the gas-tight housing, and a control device operatively controlling the free movement of the DIC optical-path apparatus; whereby the cells may be directly observed or monitored in-situ in the incubator in a three-dimensional way.

### BACKGROUND OF THE INVENTION

In the research of bio-medical field, it is always necessary to monitor or observe the cell growth conditions, cell structures, cell proliferation, or cell culture liquid by means of biological microscopes such as phase contrast or differential interference contrast (DIC) optical systems.

Whenever removing the cells from the incubator for microscopic observation, the cells will be exposed to the external environment subjected to hazards of pollution and light, temperature and humidity fluctuations, changes of oxygen, carbon dioxide and other gaseous concentration, and other uncontrollable factors, thereby greatly influencing the reliable research of cells and cell cultures.

U.S. Pat. No. 5,985,653 disclosed an incubator apparatus for maintaining and growing biological cells in a cell growth chamber of a portable cassette without exposing the cells to the external environment, and is also configured to retrieve data from, and to store data to, a memory device carried on the cassette. Since the portable cassette is so compact, it is impossible to implement a three-dimensional biological microscope such as DIC optical system in such an incubator apparatus and thereby possibly affecting efficient monitoring, observing and recording of the cell growth phenomena.

Taking an example for growing human cells, for instance, human intervertebral cells for implantation, a three-dimensional human disc cell culture can be established containing human intervertebral disc cells embedded in a carrier material forming a three-dimensional structure. The human disc cells seeded in the three-dimensional structure are capable of proliferating within the three-dimensional structure. After being cultured for a time period, the three-dimensional cell culture contains at least a portion of human intervertebral disc cells produced by cell proliferation within the three-dimensional structure. By so doing, it is very important to monitor or observe the cell growth in a three-dimensional way.

However, there is lacking of any optical monitoring subsystem provided with a three-dimensional mechanism for observing, photographing, or monitoring the cell growth or cell culture in-situ in an incubator in a three-dimensional way, thereby affecting the convenient or reliable study of animal or human living cells.

The present inventor has found the drawbacks of the conventional art and invented the present optical monitoring subsystem capable of three-dimensional monitoring or observation of cells or cell cultures directly in an incubator.

### SUMMARY OF THE INVENTION

The object of the present invention is to provide an optical monitoring subsystem including: a differential interference contrast (DIC) optical-path apparatus having a CCD camera and enclosed in a gas-tight housing stored in an incubator and a three-dimension electric driving device operatively moving along three axes of three-dimension coordinates for conveniently observing, monitoring or photographing cells in a cell specimen placed in a concave platform as recessed in the gas-tight housing, and a control device operatively controlling the free movement of the DIC optical-path apparatus; whereby the cells may be directly observed or monitored in-situ in the incubator in a three-dimensional way.

### BRIEF DESCRIPTION OF THE DRAWINGS

FIG. 1 shows a DIC optical path as applied in the present invention.

FIG. 2 is an exploded view of the present invention.

FIG. 3 is an illustration showing the overall system of the present invention.

### DETAILED DESCRIPTION

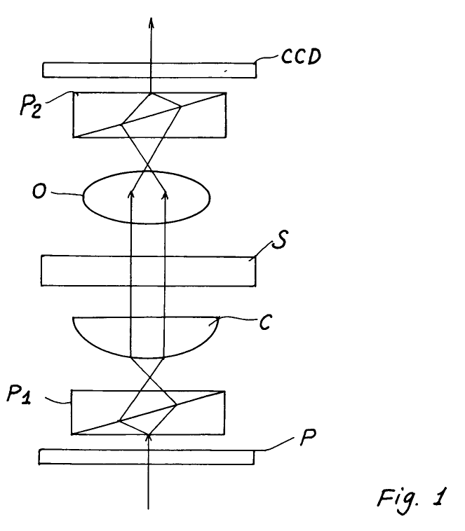

As shown in FIG. 1, a differential interference contrast (DIC) optical path generally comprises: a polarizer (P) for polarizing the light wave from a light source to be in a same polarizing angle, a beam splitter (P1) for splitting the light waves into two rays polarized at a right angle to each others, a condenser (C) for focusing the rays to pass through the specimen (S) where the length of one light wave is shorter than that of the other light wave, a beam analyzer (P2) positioned above an objective (O) for focusing the rays traveling through the objective (O) above the specimen (S) for recombining the two rays to cause interference to generate an image, and a CCD camera (CCD) for photographing or retrieving the image through the beam analyzer (P2), whereby the image thus obtained will be transmitted to a computer for processing, recording or storing of the image.

The DIC optical path is so conventional and will not be further described in detail in the present invention.

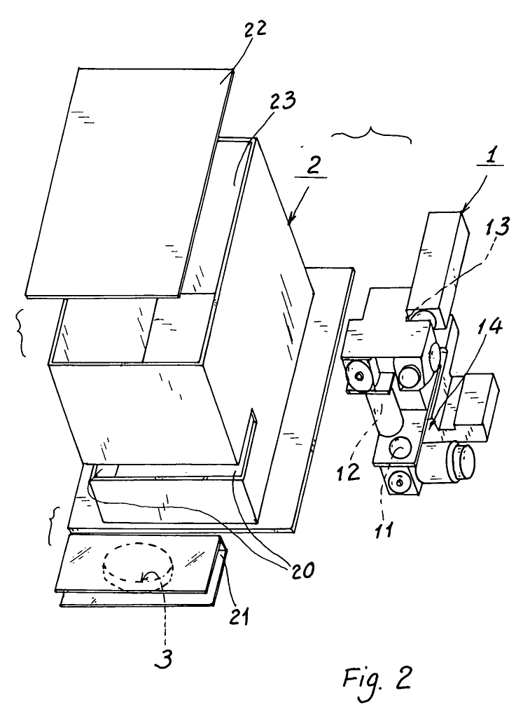

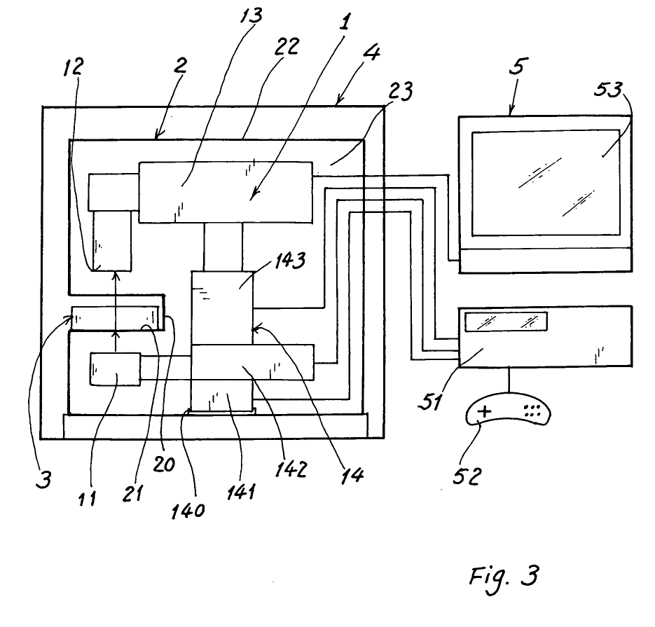

As shown in FIGS. 2 and 3, the major elements of the present invention includes: a differential interference contrast (DIC) optical-path apparatus 1; a gas-tight housing 2, both put in an incubator 4 (FIG. 3) for in-situ monitoring or observation of cells or cell cultures in a specimen 3 which is placed in a concave platform 21 as embedded or fixed in a recess 20 formed in the housing 2; and a control means (or device) 5 electrically connected with the differential interference contrast (DIC) optical-path apparatus 1.

The differential interference contrast optical-path apparatus 1 includes: a light source 11 which may be a light emitting diode (LED) for projecting light upwardly, an objective 12 projectively optically aligned with the light source 11, a CCD (charge-coupled device) camera 13, and a three-dimension electric driving device 14.

The gas-tight housing 2 includes a recess 20 formed in a front portion of the housing 2, a concave platform 21 made of transparent material and sealably fixed in the recess 20 in the housing 2 for passing light waves or rays from the light source 11 through a specimen 3 towards the objective 12 of the optical-path apparatus 1 (FIG. 3), an interior 23 defined in the housing 2 for accommodating the elements of the differential interference contrast optical-path apparatus 1 in the interior 23 of the housing 2, and a cover 22 for covering the housing 2 for storing the elements of the optical-path apparatus 1 in the interior 23 in a gas-tight, water-proof and vapor-proof situation, namely precluding or insulating entrance of moisture, vapor, air or gases into the interior 23 of the housing 2 for preventing corrosion or erosion of the elements of the optical-path apparatus 1 in order for prolonging the service life and operation precision or reliability of the present invention.

The specimen 3 of cells or cell cultures is placed on the concave platform 21 of the housing 2 for a reliable in-situ monitoring or observation of cells or cultures directly in the incubator 4, since the housing 2, the optical-path apparatus 1 and the specimen 3 are all together kept in the incubator 4 and the variables of temperature, humidity, air or gas concentration are also optimumly controlled within the incubator 4.

The control means 5 includes a control box 51 electrically connected to the three-dimension electric driving device 14, a controller 52 electrically connected to the control box 51 and operatively controlled or operated by a user, and a monitor 53 electrically connected with the camera 13 for displaying, observing or monitoring the image of the cells or cell cultures through the differential interference contrast optical-path apparatus 1.

The light source is generally given with a numeral of “11”, which actually includes a LED lamp, a polarizer, a beam splitter, and a condenser of a DIC optical path to be positioned under the specimen 3.

The objective, as shown in FIG. 3, is generally given with a numeral “12”, which actually includes the objective (or object lens) and a beam analyzer to be positioned above the specimen 3.

The CCD camera 13 is mounted on the three-dimension electric driving device 14 of the optical-path apparatus 1 for retrieving or photographing the image of the specimen 3 through the objective 12; and the light source 11 is also mounted on the three-dimension electric driving device 14. Upon moving of the objective 12, camera 13 and light source 11 as controlled by the three-dimension electric driving device 14, the cells or cell cultures in the specimen 3 will be instantly observed or monitored in situ (without being moved) on the concave platform 21, thereby preventing any unexpected vibration or movement of the specimen and thereby ensuring a reliable cell monitoring or observation in accordance with the present invention.

The three-dimension electric driving device 14 of the optical-path apparatus 1 includes a X-axis stage 141 operatively moving horizontally on a base 140 along a X-axis of three-dimension coordinates as set, preset or recorded in the control box 51 of the control means 5, a Y-axis stage 142 operatively horizontally moving on the X-axis stage 141 along a Y-axis of the three-dimension coordinates as set in the control box 51, having the light source 11 secured to the Y-axis stage 142, and a Z-axis stage 143 mounted on the Y-axis stage 142 for operatively vertically raising or descending the objective 12 and the CCD camera 13 mounted on the Z-axis stage 143 along a Z-axis of the three-dimension coordinates as set in the control box 51, whereby upon operation of the controller 52 with the control box 51 to control the three dimensional movements of the three-dimension electric driving device 14, the specimen 3 put on the concave platform 21 will be observed, monitored or photographed in a three dimensional way upon movement of the light source 11, the objective 12 and camera 13 in three dimensional orientations.

Other modifications for arranging or laying out the stages 141, 142, 143 and the DIC optical-path apparatus 1 may be further made in the present invention.

Therefore, the present invention provides a method for monitoring the cells in a three dimensional way, being beneficial for simultaneously tracking multiple targets in multiple directions and for clearly observing the cells with different vision depths.

Since the monitoring or observation is done in situ in the incubator 4, the following advantages can be achieved: prevention for environmental pollution; enhancing the cell activity; increase of operational reliability; and minimizing cell consumption in laboratory research or test for saving cost.

The DIC system of the present invention can be observed, without being inverted, in an upright way for a more convenient and reliable monitoring operation.

The image as taken may be directly displayed on the monitor 53 of the control means 5. Also, the image as taken by the CCD camera 13 may be input into a computer for recording, copying, image retrieval or any other processing jobs.

The present subsystem may be connected in series to a main system or a central control system for better management or broader applications.

The present invention may be especially recommended for monitoring or observing the cells grown in three dimensional way, thereby being beneficial for the research and practical uses in modern bio-medical fields such as for producing human cells grown in a three dimensional structure adapted for implantation use.

The present invention may be further modified without departing from the spirit and scope of the present invention.

I claim:

1. An optical monitoring subsystem comprising:
a differential interference contrast (DIC) optical-path apparatus including a light source for projecting light upwardly, an objective for receiving light from the light source through a specimen, a charge-coupled-device (CCD) camera for retrieving or photographing image of cells or cell cultures in the specimen and a three-dimension electric driving device for operatively driving the light source, the objective and the CCD camera in a three-dimensional way;
a gas-tight housing laid in an incubator for storing the differential interference contrast optical-path apparatus in said housing and having a transparent concave platform recessed in said housing for keeping the specimen in said concave platform to be monitored or observed by said DIC optical-path apparatus; and
a control means electrically connected with said DIC optical-path apparatus and operatively controlling said three-dimension electric driving device for three-dimensional movements of said light source, said objective and said CCD camera for a three-dimensional in-situ monitoring or observing of cells or cell cultures in said specimen in the incubator.

2. An optical monitoring subsystem according to claim 1, wherein said three-dimension electric driving device includes a X-axis stage operatively controlled by said control means for horizontal moving on a base along a X-axis of three-dimension coordinates as set in said control means, a Y-axis stage operatively controlled by said control means for horizontal moving on said X-axis stage along a Y-axis of the three-dimension coordinates as set in said control means, and a Z-axis stage operatively controlled by said control means for vertical moving on said Y-axis stage along a Z-axis of the three-dimension coordinates as set in said control means; said light source secured to said Y-axis stage; said objective and said CCD camera mounted on said Z-axis stage; whereby said objective is projectively optically aligned with said light source for receiving light as projected from said light source and passing through said specimen.

3. An optical monitoring subsystem according to claim 1, wherein said gas-tight housing includes a cover for sealably covering an interior in said housing for precluding vapors or moisture in the incubator from entering into the interior of the housing.

4. An optical monitoring subsystem according to claim 1, wherein said control means includes a control box electrically connected with said three-dimension electric driving device, a controller electrically connected with said control box for operatively driving said electric driving device through said control box, and a monitor electrically connected with said CCD camera for displaying image as retrieved by said CCD camera.

5. An optical monitoring subsystem according to claim 4, wherein said control means further includes a computer electrically connected with said CCD camera for retrieving, processing or recording image as taken by said CCD camera.
